(19) United States
(12) Patent Application Publication
(10) Pub. No.: US 2024/0391994 A1
(43) Pub. Date: Nov. 28, 2024

(54) ANTI-IL-23P19 ANTIBODY REGULATION OF GENES INVOLVED IN BOWEL URGENCY IN ULCERATIVE COLITIS

(71) Applicant: Eli Lilly and Company, Indianapolis, IN (US)

(72) Inventor: Mingyang SHAN, Indianapolis, IN (US)

(21) Appl. No.: 18/696,001

(22) PCT Filed: Sep. 30, 2022
(86) PCT No.: PCT/US2022/077345

§ 371 Date: Mar. 27, 2024

Related U.S. Application Data
(60) Provisional application No. 63/250,596, filed on Sep. 30, 2021.

(51) Int. Cl. C07K 16/24; A61K 9/00; A61K 39/00; A61P 1/04; G01N 33/53; G01N 33/68

(52) U.S. Cl. CPC C07K 16/244; A61K 9/0019; A61P 1/04; G01N 33/53; G01N 33/6893; A61K 2039/505; A61K 2039/545; G01N 2333/4727; G01N 2333/4737; G01N 2800/065; G01N 2800/50; G01N 2800/52

(57) ABSTRACT

The present disclosure is generally relates to methods of treating ulcerative colitis. The methods are particularly suitable for treating bowel urgency a specific sub-group of patients with ulcerative colitis and having bowel urgency.

### BACKGROUND OF THE DISCLOSURE

The present disclosure relates generally to medicine. More particularly, the present disclosure relates to methods of treating and identifying bowel urgency in patients having or suspected of having ulcerative colitis. The methods are also useful for determining the patient's response to treatment.

Ulcerative colitis (UC) is a chronic relapsing immune-mediated inflammatory bowel disease (IBD) of the colon and rectum. UC is characterized by mucosal inflammation of the colon leading to symptoms of diarrhea, rectal bleeding, and bowel urgency. While patients often experience all of these concurrently, bowel urgency is a distinct and highly disruptive symptom that is frequently overlooked by health care providers or conflated with stool frequency (SF) or diarrhea. Stool frequency refers specifically to the number of trips to the bathroom that involve the passage of stool, mucus, and/or blood over a given period of time and diarrhea refers to loose, watery stools. Bowel urgency refers to the sudden and immediate need to have a bowel movement. The conflation of these terms can lead to confusion on the part of both health care providers and patients, and mask the impact of bowel urgency on patients with UC.

More than 75-90% of patients with UC report experiencing bowel urgency. Up to half of patients with UC report at least one instance of bowel urgency per day. Patients report that bowel urgency is an important symptom of UC, distinct from stool frequency and rectal bleeding. In fact, in a recent survey bowel urgency was ranked most relevant among a list of attributes for inflammatory bowel disease (IBD) treatment decisions, and patients with UC consider bowel urgency to be a more relevant and important symptom than rectal bleeding or stool frequency. The mechanism(s) underlying bowel urgency have not been fully elucidated; however, bowel urgency has been associated with chronic inflammation, which causes changes in smooth muscle tone, hypersensitivity, and increased contractile responses in the rectum, as well as the development of submucosal fibrosis. Despite the prevalence of bowel urgency and its importance to patients with UC, there is a dearth of information on impact of this symptom on quality of life (QoL) and clinical outcomes, and bowel urgency has not been an outcome typically included in clinical trials.

Mirikizumab (LY3074828) is a humanized immunoglobulin G4 (IgG4)-variant monoclonal antibody that specifically binds to the p19 subunit of IL-23, and thus does not target IL-12, which shares a common p40 subunit with IL-23. IL-23 receptor engagement leads to activation of JAKs (mainly TYK2 and JAK2) and signal transducer and activator of transcription 3 and 4 (STAT3 and STAT4), triggering transcription of downstream target genes. IL-23 promotes the differentiation, maintenance and stabilization of pathogenic T-cell lineages, including populations that simultaneously produce multiple pro-inflammatory cytokines, such as interferon-Y, IL-17A, IL-17F and IL-22, as well as activation and induction of effector function of colitogenic innate lymphoid cells.

Substantial morbidity and impaired quality of life results from typical symptoms such diarrhea, rectal bleeding, and urgency. Treatment aims include achieving symptom control (clinical remission), suppressing intestinal inflammation leading to mucosal healing (endoscopic remission), and preserving gut functionality. Current treatment options include 5-aminosalicylates, glucocorticoids, thiopurines, the Janus-associated kinase (JAK) inhibitor tofacitinib, and biologics that antagonize TNFα, the p-40 subunit of IL-12/IL-23, and a4b7 integrin. However, up to one third of patients do not respond to induction treatment and approximately 40% of patients who initially benefited subsequently lose response. It was recently shown that anti-TNF therapy is associated with potentially serious adverse effects. A new class of biologics that block integrin signaling, thereby reducing lymphocyte trafficking to the intestinal mucosa and reducing mucosal inflammation, represent a more favorable safety profile. In a recent trial vedolizumab, an α4β7 integrin blocker, was shown to be more effective than the TNF inhibitor adalimumab for moderate-to-severe UC.

Interleukin-23 (IL-23) is a novel therapeutic target in IBD, a heterodimeric cytokine composed of a p19 subunit and a p40 subunit that it shares with IL-12. IL-23 receptor engagement leads to activation of JAKs (mainly TYK2 and JAK2) and signal transducer and activator of transcription 3 and 4 (STAT3 and STAT4), triggering transcription of downstream target genes. IL-23 promotes the differentiation, maintenance and stabilization of pathogenic T-cell lineages, including populations that simultaneously produce multiple pro-inflammatory cytokines, such as interferon-γ, IL-17A, IL-17F and IL-22, as well as activation and induction of effector function of colitogenic innate lymphoid cells. Therapeutic blockade of p40 is effective in both UC and CD, and drugs targeting p19 are being studied for both UC and CD.

Data from single cell RNASeq studies have suggested that inflamed mucosal fibroblasts, tissue resident monocytes, and dendritic cells are enriched in anti-TNF-resistant (TNF^{R}) therapy compared to UC patients who respond to anti-TNFs. In these studies, Smillie et al. scored cell subsets for gene signatures of TNF^{R} and sensitivity based on a meta-analysis of bulk expression data from 60 responders and 57 non-responders to anti-TNF therapy. TNF^{R} was strongly associated with genes enriched in immune associated fibroblasts (IAFs), inflammatory monocytes, and DC2 cells. In contrast, favourable response to anti-TNF therapy was evident in the transcriptome signature in epithelial cells, which represents healthy mucosa prevalent in UC patients in remission.

There remains a need for alternative compositions and methods to diagnose and treat symptoms of ulcerative colitis such as bowel urgency.

### BRIEF DESCRIPTION OF THE DISCLOSURE

The present disclosure is generally relates to methods of treating and diagnosing patients with ulcerative colitis. The methods are particularly suitable for treating a specific sub-group of patients with ulcerative colitis. The methods are particularly suitable for treating and identifying bowel urgency in patients having or suspected of having ulcerative colitis.

In one aspect, the present disclosure is directed to a method of treating bowel urgency in a patient having or suspected of having ulcerative colitis. The method includes: obtaining a first sample from the patient; analyzing the first sample to detect at least one biomarker selected from C-reactive protein (CRP) and fecal calprotectin (fCLP); administering an anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to a method of identifying a patient having or suspected of having ulcerative colitis as a candidate patient for receiving an anti-IL-23p19 antibody treatment for bowel urgency. The method includes: obtaining a sample from the patient; analyzing the sample for at least one biomarker selected from C-reactive protein (CRP) and fecal calprotectin (fCLP); and identifying the patient as a candidate patient for receiving the anti-IL-23p19 antibody treatment for bowel urgency based on the analysis of the at least one biomarker.

In another aspect, the present disclosure is directed to a method of identifying a patient having or suspected of having ulcerative colitis as a candidate patient for receiving an anti-IL-23p19 antibody treatment for bowel urgency. The method includes: obtaining a sample from the patient; analyzing the sample for at least one biomarker selected from C-reactive protein (CRP) and fecal calprotectin (fCLP); and identifying the patient as a candidate patient for receiving the anti-IL-23p19 antibody treatment for bowel urgency based on the analysis of the at least one biomarker.

In another aspect, the present disclosure is directed to a method for identifying risk for bowel urgency in a patient having or suspected of having ulcerative colitis. The method includes: (a) determining an expression level of at least one biomarker selected from C-reactive protein (CRP) and fecal calprotectin (fCLP) in a sample obtained from the patient; (b) comparing the determined expression level of the at least one biomarker to a reference expression level of at least one biomarker selected from C-reactive protein (CRP) and fecal calprotectin (fCLP); and (c) identifying that the patient is at risk for bowel urgency if the biomarker expression level in the patient is changed as compared to the reference expression level. In one embodiment, the patient is identified as being at risk for bowel urgency if the biomarker from the patient is changed as compared to the reference expression level. In another embodiment, the patient is identified as not being at risk for bowel urgency if the biomarker is unchanged as compared to the reference expression level.

In another aspect, the present disclosure is directed to a method of determining a reduction in bowel urgency in a patient having or suspected of having ulcerative colitis in response to an anti-IL-23p19 antibody treatment. The method includes analyzing a sample obtained from a patient before the patient receives an anti-IL-23p19 antibody treatment for at least one biomarker including C-reactive protein (CRP) and fecal calprotectin (fCLP); analyzing a sample obtained from a patient after the patient receives an anti-IL-23p19 antibody treatment for at least one biomarker including C-reactive protein (CRP) and fecal calprotectin (fCLP); and determining that the bowel urgency patient having or suspected of having ulcerative colitis is reduced in response to the anti-IL-23p19 antibody treatment if a change in expression level in the at least one biomarker after the patient receives the anti-IL-23p19 antibody treatment is detected.

In another aspect, the present disclosure is directed to an anti-IL-23p19 antibody for use in the treatment of bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to an anti-IL-23p19 antibody for use in reducing bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to use of an anti-IL-23p19 antibody in the manufacture of a medicament for use in the treatment of bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to use of an anti-IL-23p19 antibody for the manufacture of medicament for use in reducing bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to a biomarker panel. The biomarker panel includes at least one biomarker including C-reactive protein (CRP), fecal calprotectin (fCLP), C-X-C Motif Chemokine Ligand 8 (CXCL8; also interleukin 8 and IL-8), Aquaporin 9 (AQP9), Interleukin 1 beta (ILIB), S100 Calcium Binding Protein A9 (S100A9), Triggering Receptor Expressed on Myeloid Cells 1 (TREM1), Matrix Metallopeptidase 12 (MMP12), Matrix Metallopeptidase 1 (MMP1), Matrix Metallopeptidase 7 (MMP7), Transcobalamin 1 (TCN1), Dual Oxidase 2 (DUOX2), Dual Oxidase Maturation Factor 2 (DUOXA2), Solute Carrier Family 6 Member 14 (SLC6A14), Vanin 1 (VNN1), ΔTP Binding Cassette Subfamily A Member 12 (ABCA12), Regenerating Family Member 1 Beta (REG1B), Complement Component 4 Binding Protein Alpha (C4BPA), Guanylate Cyclase Activator 2B (GUCA2B), Otopetrin 2 (OTOP2), Aquaporin 8 (AQP8), Solute Carrier Family 26 Member 2 (SLC26A2), Alcohol Dehydrogenase 1C (Class I), Gamma Polypeptide (ΔDH1C), Matrix Metallopeptidase 3 (MMP3), Regenerating Family Member 3 Alpha (REG3A), Deleted In Malignant Brain Tumors 1 (DMBT1), Regenerating Family Member 1 Gamma, Pseudogene (REG1P), S100 Calcium Binding Protein A8 (S100A8), Immunoglobulin Kappa Variable 2D-40 (IGKV2D-40), Peptidase Inhibitor 3 (PI3), Tumor Necrosis Factor Alpha Induced Protein 3 (TNIP3), Regenerating Family Member 1 Alpha (REG1A), Indoleamine 2,3-Dioxygenase 1 (IDO1), Nitric Oxide Synthase 2 (NOS2), Matrix Metallopeptidase 10 (MMP10), C-X-C Motif Chemokine Ligand 1 (CXCL1), Prostaglandin E Synthase 2 (PTGS2), ΔTP Binding Cassette Subfamily G Member 2 (Junior Blood Group) (ABCG2), 3-Hydroxy-3-Methylglutaryl-CoA Synthase 2 (HMGCS2), Transmembrane And Immunoglobulin Domain Containing 1 (TMIGD1), Guanylate Cyclase Activator 2A (GUCA2A), Long Intergenic Non-Protein Coding RNA 2023 (LOC101928405), Membrane Spanning 4-Domains A12 (MS4A12), UDP Glucuronosyltransferase Family 2 Member A3 (UGT2A3), Transient Receptor Potential Cation Channel Subfamily M Member 6 (TRPM6), Neurexophilin And PC-Esterase Domain Family Member 4 (NXPE4), Solute Carrier Family 16 Member 9 (SLC16A9), Phosphoenolpyruvate Carboxykinase 1 (PCK1), ENSG00000266446 Novel Transcript, Sense Intronic CDKN2B-AS1 (CDKN2B-AS1), Transmembrane Protein 236 (TMEM236), CD177 Molecule Pseudogene 1 (CD177P1), Solute Carrier Family 17 Member 4 (SLC17A4), and Zymogen Granule Protein 16 (ZG16).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 depicts the relative importance of urgency status or Mayo subscores to variation in Inflammatory Bowel Disease Questionnaire (IBDQ) measures at Week 12. Partial R² values were calculated from an ANCOVA model that includes bowel urgency, rectal bleeding, or stool frequency status, to compare the proportion of variation in QoL gained from the addition of each symptom into the model. Brighter green indicates a stronger contribution while red indicates a weaker contribution.

FIGS. 2A-2D depict the change from baseline CRP or fCLP in patients with or without bowel urgency. Mean change from baseline±SE of CRP and fCLP at Week 12 (FIGS. 2A-2B) and Week 52 (FIGS. 2C-2D) in patients with presence of urgency or absence of urgency at those time points.

FIGS. 3A and 3B depict the time to symptomatic response and remission in patients with or without bowel urgency. Kaplan-Meier analyses demonstrate how time to achieving symptomatic remission (RB=0, SF=0 or 1; FIG. 2A) or symptomatic response (≥35% decrease from baseline with either a RB decrease of ≥1 or RB=0 or 1; FIG. 2B) changes as patients achieve absence of urgency (blue line) at baseline or Weeks 4, 8, and 12.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, “a subject in need thereof” refers to a subject having, suspected of having, susceptible to and at risk of a specified disease, disorder, or condition. More particularly, in the present disclosure the methods of treating bowel urgency in patients having or suspected of having ulcerative colitis and the methods of screening biomarkers is to be used with a subset of subjects who have, are suspected of having, are susceptible to and are at elevated risk for experiencing bowel urgency with ulcerative colitis. Such subjects may include, but are not limited to, subjects having, suspected of having, susceptible to and at risk of bowel urgency with ulcerative colitis. Subjects having, suspected of having, susceptible to and at risk of bowel urgency with ulcerative colitis due to family history, age, environment, and/or lifestyle.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects in need of treatment described herein.

As used herein, “susceptible” and “at risk” refer to having little resistance to a certain disease, disorder or condition, including being genetically predisposed, having a family history of, and/or having symptoms of the disease, disorder or condition.

As used herein, the terms “treating,” “treat,” or “treatment,” refer to restraining, slowing, lessening, reducing, or reversing the progression or severity of an existing symptom, disorder, condition, or disease, or ameliorating clinical symptoms and/or signs of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Those in need of treatment include those already with the disease.

As used herein, the term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a human IL-23. For example, antibody fragments capable of binding to IL-12/23 or portions thereof, including, but not limited to, Fab (e.g. by papain digestion), Fab′ (e.g., by pepsin digestion and partial reduction) and F(ab′) 2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g. by molecular biology techniques) fragments, are encompassed by the present invention (see, e.g. Colligan et al., Current Protocols in Immunology, John Wiley & Sons, NY, NY, (1994-2001)). Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′) 2 heavy chain portion can be designed to include DNA sequences encoding the CHI domain and/or hinge region of the heavy chain. The various portions of antibodies can be joined together chemically by conventional techniques, or can be prepared as a contiguous protein using genetic engineering techniques.

As used herein “anti-IL-23p19 antibody” refers to an antibody that binds to the p19 subunit of human IL-23 but does not bind to the p40 subunit of human IL-23. An anti-IL-23p19 antibody thus binds to human IL-23 but does not bind to human IL-12. Suitable examples of anti-IL-23p19 antibodies that may be used in the methods of the present invention include guselkumab, tildrakizumab, risankizumab, mirikizumab and brazikumab. Guselkumab, CAS Registry No. 1350289-85-8, is a fully human IgG₁ lambda monoclonal antibody that binds to the p19 subunit of human IL-23. The antibody and methods of making same are described in U.S. Pat. No. 7,935,344. Tildrakizumab, CAS Registry No. 1326244 October 3, is a humanized, IgG1 kappa monoclonal antibody targeting the p19 subunit of human IL-23. The antibody and methods of making same are described in U.S. Pat. No. 8,293,883. Risankizumab, CAS Registry No. 1612838-76-2, is a humanized, IgG1 kappa monoclonal antibody targeting the p19 subunit of human IL-23. The antibody and methods of making same are described in U.S. Pat. No. 8,778,346. Mirikizumab, CAS Registry No. 1884201-71-1, is a humanized, IgG4-kappa monoclonal antibody targeting the p19 subunit of human IL-23. The antibody and methods of making same are described in U.S. Pat. No. 9,023,358. Brazikumab, CAS Registry No. 1610353-18-8, is a humanized, IgG2-lambda monoclonal antibody targeting the p19 subunit of human IL-23. The antibody and methods of making same are described in U.S. Pat. No. 8,722,033.

The anti-IL-23p19 antibody, or pharmaceutical compositions comprising the same, may be administered by parenteral routes (e.g., subcutaneous, intravenous, intraperitoneal, intramuscular, or transdermal). Pharmaceutical compositions comprising an anti-IL-23p19 antibody for use in the methods of the present invention can be prepared by methods well known in the art (e.g., Remington: The Science and Practice a Pharmacy, 19^{th} edition (1995), (A. Gennaro et al., Mack Publishing Co.) and comprise an antibody as disclosed herein, and one or more pharmaceutically acceptable carriers, diluents, or excipients.

As used herein, the term “biomarker” refers to any molecule or group of molecules found in a biological sample that can be used to characterize the biological sample or a subject from which the biological sample is obtained. For example, a biomarker may be a molecule or group of molecules whose presence, absence, or relative abundance is: characteristic of a particular cell or tissue type or state; and/or characteristic of a particular pathological condition or state; and/or indicative of the severity of a pathological condition, the likelihood of progression or regression of the pathological condition, and/or the likelihood that the pathological condition will respond to a particular treatment. As another example, the biomarker may be a cell type or a microorganism (such as a bacterium, mycobacterium, fungus, virus, and the like), or a substituent molecule or group of molecules thereof. Biomarkers provided herein can be diagnostic biomarkers that can be used to detect and/or confirm the presence of ulcerative colitis. Biomarkers provided herein can also be monitoring biomarkers that can be serially analyzed to assess the status of ulcerative colitis. Biomarkers provided herein can also be pharmacodynamic biomarkers that can be used to determine a patient's response to an anti-IL-23p19 antibody treatment. Biomarkers provided herein can also be predictive biomarkers that can be used to predict or identify an individual or group of individuals more likely to experience a favorable or unfavorable effect from an anti-IL-23p19 antibody treatment. Biomarkers provided herein can also be safety biomarkers that are measured before and/or after anti-IL-23p19 antibody administration to indicate the likelihood, presence, or extent of a toxicity to anti-IL-23p19 antibody. Biomarkers provided herein can also be prognostic biomarkers to identify ulcerative colitis progression and/or recurrence. Biomarkers provided herein can also be susceptibility/risk biomarkers that can indicates the potential for an individual to develop bowel urgency with ulcerative colitis but who has not been diagnosed as having and/or has not experienced bowel urgency with ulcerative colitis. Biomarkers provided herein can also be surrogate biomarkers that explain the clinical outcome following anti-IL-23p19 antibody treatment.

As used herein, “expression level of a biomarker” refers to the process by which a gene product is synthesized from a gene encoding the biomarker as known by those skilled in the art. The gene product can be, for example, RNA (ribonucleic acid) and protein. Expression level can be quantitatively measured by methods known by those skilled in the art such as, for example, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, enzyme linked immunosorbent assay (ELISA), and combinations thereof.

As used herein, “a reference expression level” of a biomarker refers to the expression level of a biomarker established for a subject without bowel urgency who has ulcerative colitis, expression level of a biomarker in a normal/healthy subject without bowel urgency and without ulcerative colitis as determined by a medical professional and/or research professional using established methods as described herein, and/or a known expression level of a biomarker obtained from literature. The reference expression level of the biomarker can also refer to the expression level of the biomarker established for any combination of subjects such as a subject with ulcerative colitis but without bowel urgency, a subject without ulcerative colitis, expression level of the biomarker in a normal/healthy subject without ulcerative colitis, and expression level of the biomarker for a subject without bowel urgency but having ulcerative colitis at the time the sample is obtained from the subject and who later exhibits bowel urgency with ulcerative colitis. The reference expression level of the biomarker can also refer to the expression level of the biomarker obtained from the subject to which the method is applied. As such, the change within a subject from visit to visit can indicate an increased or decreased risk for bowel urgency with ulcerative colitis. For example, a plurality of expression levels of a biomarker can be obtained from a plurality of samples obtained from the same subject and used to identify differences between the pluralities of expression levels in each sample. Thus, in some embodiments, two or more samples obtained from the same subject can provide an expression level(s) of a blood biomarker and a reference expression level(s) of the blood biomarker. The reference expression level can also refer to the expression level of a biomarker in a “placebo responder”. As used herein a “placebo responder” is a subject having bowel urgency with ulcerative colitis as determined by a medical professional and/or research professional using established methods as described herein who demonstrates clinical improvement, but who is not administered an anti-IL-23p19 antibody. Without being bound by theory, it is believed that placebo responders demonstrate improvement due to lifestyle changes made by the placebo responder in response to an ulcerative colitis diagnosis and/or counseling and/or medical follow-up

A particularly suitable anti-IL-23p19 antibody is mirikizumab (also referred to herein as “miri”). Mirikizumab is a p19-directed Interleukin-23 (IL-23) monoclonal antibody.

As used herein, “dose”, “dosing” and “dosage” refers to the administration of a substance (for example, an anti-IL-23p19 antibody) to achieve a therapeutic objective (for example, the treatment of bowel urgency in patients having or suspected of having ulcerative colitis). Suitable anti-IL-23p19 antibody induction dosage includes from about 50 mg to about 600 mg. A particularly suitable dosage is a 300 mg induction dose of an anti-IL-23p19 antibody. The induction dose of an anti-IL-23p19 antibody is suitably administered intravenously. Suitably, a patient is administered 50 mg to 600 mg, preferably 300 mg of an induction dose every 4 weeks for 12 weeks. The induction dose(s) may be followed by at least one maintenance dose ranging from about 150 mg to about 400 mg, preferably 200 mg, of an anti-IL-23p19 antibody. A particularly suitable dosage is a 200 mg maintenance dose of an anti-IL-23p19 antibody. Suitably, a patient is administered 150 mg to 400 mg of a maintenance dose every 4 weeks or every 12 weeks. Administration of at least one induction dose of an anti-IL-23p19 antibody to a patient in need thereof in an induction period is intended to induce a desired therapeutic effect, the desired therapeutic effect being clinical remission, clinical response, endoscopic remission, endoscopic healing and/or symptomatic remission. If the patient achieves a desired therapeutic effect at the end of the induction period, he/she is subsequently administered at least one maintenance dose to maintain at least one of the therapeutic effect(s) obtained during the induction period, the therapeutic effect(s) being clinical remission, clinical response, endoscopic remission, endoscopic healing and/or symptomatic remission. There is no minimum or maximum duration of the induction period but it is typically 4, 8 or 12 weeks in duration, with the end of induction period being an end-of-induction assessment typically occurring 4 or 8 weeks after the last induction dose has been administered. Administration of the induction dose can be extended termed “extended induction dose” to distinguish it from the initial induction dose-if the patient does not achieve clinical response at the end of the initial induction period. If the patient achieves clinical response at the end of the extended induction period, at least one maintenance dose of the anti-IL-23p19 antibody is administered to maintain clinical response or other desired therapeutic effect(s) such as clinical remission, endoscopic remission, endoscopic healing and/or symptomatic remission. The first maintenance dose is administered 4-12 weeks after the last extended induction dose is administered to the patient. The 4-12 week period accommodates variation in the period between the administration of last extended induction dose and the end of extended-induction assessment. The maintenance dose(s) are administered at 4, 8 or 12 week interval(s) after administration of the first maintenance dose. Maintenance dose(s) can be administered by subcutaneous injection. If the patient develops a loss of response during the maintenance period, one, two or three rescue dose(s) of the anti-IL-23p19 antibody are administered to the patient, wherein one or more further maintenance dose(s) of the anti-IL-23p19 antibody are administered to the patient if the patient achieves clinical response 4-12 weeks after the last rescue dose is administered, wherein loss of response is defined as: (a) >2-point increase from baseline in the combined stool frequency (SF) and rectal bleeding (RB) scores (b) combined SF and RB score of >4, on 2 consecutive visits >7 days apart with confirmation of negative Clostridium difficile testing and (c) endoscopic subscore (ES) of 2 or 3, and wherein clinical response is defined as achieving a decrease in the 9 point Modified Mayo Score (MMS) subscore of >2 points and >30-35% from baseline, with either a decrease of rectal bleeding (RB) subscore of >1 or a RB subscore of 0 or 1.

The methods disclosed herein can further include obtaining three or more samples from the patient. It is particularly suitable to obtain multiple samples from a patient, a reference subject, and a placebo responder for analysis of samples to determine whether expression levels of biomarkers change, remain changed over time, are maintained over time, and the like.

Suitable samples include whole blood, plasma, serum, tissue biopsy, fecal samples, and combinations thereof. Particularly suitable tissue biopsy samples include biopsy obtained from the edge of ulcers, biopsy obtained from the edge of erosions, biopsies obtained spaced throughout affected mucosa, and combinations thereof.

Samples are obtained at about 4 weeks following anti-IL-23p19 antibody administration, at about 12 weeks following anti-IL-23p19 antibody administration, at about 52 weeks following anti-IL-23p19 antibody administration, and combinations thereof. Samples can further be obtained after 52 weeks following anti-IL-23p19 antibody administration. Samples can further be obtained at other intervals including daily, weekly, monthly, and yearly.

Expression (and expression level) can be determined by microarray analysis. Other suitable methods for determining expression include amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, and combinations thereof.

The methods disclosed herein can further include analyzing a tissue sample using histopathology. Tissue samples can be processed and stained for bright field microscopy using H & E stain, Romanowsky staining, and even unstained tissue samples. Tissue samples can also be stained using an antibody that specifically binds to a biomarker to be detected. The antibody can include a label such as a fluorescent label and the tissue can be examined by exposing the tissue sample to ultraviolet light. The biomarker antibody can be directly labeled with a fluorescent label or detected using a fluorescently labeled second antibody that specifically binds the biomarker antibody. Tissue samples can be labeled to detect a single biomarker or multiple biomarkers. Tissue samples can also be analyzed using spatial transcriptomics to determine subcellular localization of the biomarker mRNAs.

In one aspect, the present disclosure is directed to a method of treating bowel urgency in a patient having or suspected of having ulcerative colitis. The method includes: obtaining a first sample from the patient; analyzing the first sample to detect at least one biomarker selected from C-reactive protein (CRP) and fecal calprotectin (fCLP); administering an anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to an anti-IL-23p19 antibody for use in the treatment of bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to use of an anti-IL-23p19 antibody in the manufacture of a medicament for use in the treatment of bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

Particularly suitable biomarkers include C-reactive protein (CRP), fecal calprotectin (fCLP), and combinations thereof. In one embodiment, the biomarker is decreased following the anti-IL-23p19 antibody treatment and includes at least one of C-reactive protein (CRP) and fecal calprotectin (fCLP).

In one embodiment, a change in the expression detected in the second sample from the expression detected in the first sample indicates that the anti-IL-23p19 antibody administration should be continued. A change in the expression can be a decrease in the expression in the second (or subsequent) sample as compared to the expression in the first sample. A change in the expression can also be an increase in the second (or subsequent) sample as compared to the expression in the first sample. The change in expression level in the sample(s) obtained from the patient administered the anti-IL-23p19 antibody can further be compared to one of an expression level in a sample(s) obtained from a healthy subject (a subject who is not suspected of having or has ulcerative colitis) and an expression level in a sample(s) obtained from a patient having or suspected of having ulcerative colitis who does not experience bowel urgency and is not administered an anti-IL-23p19 antibody.

In another embodiment, change in the expression level detected in the second sample from the expression level detected in the first sample indicates that the anti-IL-23p19 antibody administration should be discontinued. A change in the expression level can be an increase in the expression level in the second (or subsequent) sample as compared to the expression level in the first sample. A change in the expression level can also be a decrease in the second (or subsequent) sample as compared to the expression level in the first sample. The change in expression level in the sample(s) obtained from the patient administered the anti-IL-23p19 antibody can further be compared to one of an expression level in a sample(s) obtained from a healthy subject (a subject who is not suspected of having or has ulcerative colitis) and an expression level in a sample(s) obtained from a patient having or suspected of having ulcerative colitis who does not experience bowel urgency and is not administered an anti-IL-23p19 antibody.

In an embodiment, the sample is whole blood, plasma, serum, tissue biopsy, fecal samples, and combinations thereof.

In another embodiment, the sample is a combination of a serum sample and a fecal sample, wherein CRP is detected in the serum sample and fCLP is detected in the fecal sample.

In another embodiment, CRP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, or combinations thereof, and fCLP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunonephelometry assay, immunoprecipitation, immunohistochemistry, or combinations thereof.

In another embodiment, CRP is detected in a serum sample by a CRP HS immunonephelometry assay and fCLP is detected in a fecal samples by an ELISA.

In another embodiment, the first sample is taken before or simultaneous with administration of the anti-IL-23p19 antibody, and wherein the second sample is taken at least two weeks, at least four weeks, at least eight weeks, at least twelve weeks, at least sixteen weeks, at least twenty weeks, at least twenty-four weeks, at least twenty-eight weeks, at least thirty weeks, at least thirty-two weeks, at least thirty-six weeks, at least forty weeks, at least forty-four weeks, at least forty-eight weeks, or at least fifty-two weeks, after the first administration of the anti-IL-23p19 antibody.

In another embodiment, the anti-IL-23p19 antibody is mirikizumab, guselkumab, risankizumab, tildrakizumab or brazikumab.

In a preferred embodiment, the anti-IL-23p19 antibody is mirikizumab.

In another embodiment, administering mirikizumab to the patient comprises:
a) administering at least one induction dose of mirikizumab to the patient, wherein the induction dose comprises 50 mg to 600 mg of mirikizumab; and
b) administering at least one maintenance dose(s) of mirikizumab to the patient after the last induction dose is administered, wherein the maintenance dose comprises 150 to 400 mg of mirikizumab.

In another embodiment, the induction doses of mirikizumab are administered about every 4 weeks for about 12 weeks.

In another embodiment, the induction dose(s) of mirikizumab is administered intravenously.

In another embodiment, the at least one maintenance dose is administered subcutaneously.

In another embodiment, the method/treatment comprises:
a) administering three induction doses of mirikizumab to the patient by intravenous infusion at 4-week intervals, wherein each induction dose comprises 300 mg of mirikizumab; and
b) administering maintenance doses of mirikizumab to the patient by subcutaneous injection at 4 week or 12 week intervals, wherein the first maintenance dose is administered 2-8 weeks after the last induction dose is administered and wherein each maintenance dose comprises 200 mg of mirikizumab.

In another embodiment, the first maintenance dose is administered 4-6 weeks after the last induction dose is administered.

In another embodiment, subsequent maintenance dose(s) of mirikizumab are administered at 4-week intervals after administration of the first maintenance dose.

In another embodiment, subsequent maintenance dose(s) of mirikizumab are administered at 12-week intervals after administration of the first maintenance dose.

In an alternative preferred embodiment, the anti-IL-23p19 antibody is guselkumab.

In another embodiment, the method/treatment comprises:
a) administering three induction doses of guselkumab to the patient by intravenous infusion at 4-week intervals, wherein each induction dose comprises 100-500 mg of guselkumab; and
b) administering maintenance doses of guselkumab to the patient by subcutaneous injection at 2-week, 4-week, 6-week, 8-week or 12-week intervals, wherein the first maintenance dose is administered 2-8 weeks after the last induction dose is administered.

In another embodiment, each induction dose comprises 200 mg of guselkumab.

In another embodiment, each induction dose comprises 400 mg of guselkumab.

In an alternative embodiment, the anti-IL-23p19 antibody is risankizumab.

In an alternative embodiment, the anti-IL-23p19 antibody is tildrakizumab.

In an alternative embodiment, the anti-IL-23p19 antibody is brazikumab.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Ulcerative Colitis Disease Activity Index (UCDAI), Geboes Score, Robarts Histopathology Index (RHI), and combinations thereof.

The Mayo score is a composite instrument comprised of the following 4 subscores:
(i) Stool Frequency (SF): The SF subscore is a patient-reported measure. This item reports the number of stools in a 24-hour period, relative to the normal number of stools for that patient in the same period, on a 4-point scale. A stool is defined as a trip to the toilet when the patient has either a bowel movement, or passes blood alone, blood and mucus, or mucus only. The total number of stools passed in a 24-hour period is recorded by the patient. The reference “normal” SF for that patient is typically recorded at the outset of a study or period of observation. Normal SF for that patient is on the reported SF when the patient was in remission or, if the patient has never achieved remission, the reported SF before initial onset of signs and symptoms of UC.

| Stool Frequency Subscore | Score |
|---|---|
| Normal number of stools for subject | 0 |
| 1 to 2 stools more than normal | 1 |
| 3 to 4 stools more than normal | 2 |
| 5 or more stools than normal | 3 |

(ii) Rectal Bleeding (RB): The RB subscore is a patient-reported measure. This item reports the most severe amount of blood passed per rectum for a given day, on a 4-point scale.

| Rectal Bleeding Subscore | Score |
|---|---|
| No blood seen | 0 |
| Streaks of blood with stool | 1 |
| less than half of the time | |
| Obvious blood (more than just streaks) or | 2 |
| streaks of blood with stool most of the time | |
| Blood alone passed | 3 |

(iii) Endoscopic Subscore(ES): The ES is a physician-reported measure that reports the worst appearance of the mucosa on flexible sigmoidoscopy or colonoscopy, on a 4-point scale. Consistent with current clinical practice, friability is excluded from the definition of an ES of 1.

| Endoscopic Subscore | Score |
|---|---|
| Normal or inactive disease | 0 |
| Mild disease (erythema, | 1 |
| decreased vascular pattern) | |
| Moderate disease (marked erythema, | 2 |
| absent vascular pattern, friability, erosions) | |
| Severe disease (spontaneous bleeding, ulceration) | 3 |

(iv) Physician's Global Assessment (PGA): The PGA is a physician-reported measure that summarizes the assessment of the patient's UC disease activity on a 4-point scale.

| Physician's Global Assessment | Score |
|---|---|
| Normal | 0 |
| Mild disease | 1 |
| Moderate disease | 2 |
| Severe disease | 3 |

TABLE 1
Modified Mayo Score
Modified Mayo Score (MMS)

| | | Rectal Bleeding | Endoscopy Score |
|---|---|---|---|
| Index | Stool Frequency (SF) | (RB) | (ES) |
| MMS | 0 = Normal number of | 0 = No blood seen | 0 = normal or |
| | stools per day for this | 1 = streaks of | inactive disease |
| | patient | blood with stool | 1 = mild disease |
| | 1 = 1 to 2 more stools | less than half the | (erythema |
| | per day for this patient | time | decreased vascular |
| | 2 = 3 to 4 more stools | 2 = obvious blood | pattern) |
| | than normal | with stool most of | 2 = moderate |
| | 3 = 5 or more stools | the time | disease (marked |
| | than normal | 3 = blood alone | erythema, absent |
| | | passed | vascular pattern, |
| | | | friability erosions) |
| | | | 3 = severe disease |
| | | | (spontaneous |
| | | | bleeding, |
| | | | ulceration) |

Each subscore is scored on a 4-point scale, ranging from 0 to 3, to give a maximum Mayo score of 12.

The MMS is a modification made to the original Mayo Index reference (Schroeder et al., New Eng J Med, 317 (26): 1625-1629, 1987) and includes 3 of the 4 subscores of the Mayo Score. It does not include the Physician's Global Assessment. The MMS evaluates three subscores, each on a scale of 0 to 3 with a maximum total score of 9. The following table summarizes the respective MMS subscales for scoring.

Patients having a Mayo Score of 6-12 or a MMS of 4-9, each with an ES of ≥2, are defined as having moderate to severely active ulcerative colitis.

In another aspect, the present disclosure is directed to a method of identifying a patient having or suspected of having ulcerative colitis as a candidate patient for receiving an anti-IL-23p19 antibody treatment for bowel urgency. The method includes: obtaining a sample from the patient; analyzing the sample for at least one biomarker selected from C-reactive protein (CRP) and fecal calprotectin (fCLP); and identifying the patient as a candidate patient for receiving the anti-IL-23p19 antibody treatment for bowel urgency based on the analysis of the at least one biomarker.

In an embodiment, the sample is whole blood, plasma, serum, tissue biopsy, fecal samples, and combinations thereof.

In another embodiment, the sample is a combination of a serum sample and a fecal sample, wherein CRP is detected in the serum sample and fCLP is detected in the fecal sample.

In another embodiment, CRP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, or combinations thereof, and fCLP is detected by in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunonephelometry assay, immunoprecipitation, immunohistochemistry, or combinations thereof.

In another embodiment, the CRP is detected in a serum sample by a CRP HS immunonephelometry assay and fCLP is detected in a fecal sample by an ELISA.

In another embodiment, the anti-IL-23p19 antibody is mirikizumab, guselkumab, risankizumab, tildrakizumab or brazikumab.

In a preferred embodiment, the anti-IL-23p19 antibody is mirikizumab.

In another aspect, the present disclosure is directed to a method for identifying risk for bowel urgency in a patient having or suspected of having ulcerative colitis. The method includes: (a) determining an expression level of at least one biomarker selected from C-reactive protein (CRP) and fecal calprotectin (fCLP) in a sample obtained from the patient; (b) comparing the determined expression level of the at least one biomarker to a reference expression level of at least one biomarker selected from C-reactive protein (CRP) and fecal calprotectin (fCLP); and (c) identifying that the patient is at risk for bowel urgency if the biomarker expression level in the patient is changed as compared to the reference expression level. In one embodiment, the patient is identified as being at risk for bowel urgency if the biomarker from the patient is changed as compared to the reference expression level. In another embodiment, the patient is identified as not being at risk for bowel urgency if the biomarker is unchanged as compared to the reference expression level.

In an embodiment, the sample is whole blood, plasma, serum, tissue biopsy, fecal samples, and combinations thereof.

In another embodiment, the sample is a combination of a serum sample and a fecal sample, wherein CRP is detected in the serum sample and fCLP is detected in the fecal sample.

In another embodiment, CRP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, or combinations thereof, and fCLP is detected by in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunonephelometry assay, immunoprecipitation, immunohistochemistry, or combinations thereof.

In another embodiment, the CRP is detected in a serum sample by a CRP HS immunonephelometry assay and fCLP is detected in a fecal sample by an ELISA.

The method can further include analyzing samples obtained before anti-IL-23p19 antibody administration and following anti-IL-23p19 antibody administration for at least one biomarker selected from C-reactive protein (CRP), fecal calprotectin (fCLP), and combinations thereof.

The methods can further include analyzing at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of reducing bowel urgency in a patient having or suspected of having ulcerative colitis. The method includes: obtaining a first sample from the patient; analyzing the sample for at least one biomarker selected from C-reactive protein (CRP) and fecal calprotectin (fCLP); administering an anti-IL-23p19 antibody to the patient; obtaining second sample from the patient; and analyzing the second sample for at least one biomarker selected from C-reactive protein (CRP) and fecal calprotectin (fCLP).

In another aspect, the present disclosure is directed to an anti-IL-23p19 antibody for use in reducing bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In another aspect, the present disclosure is directed to use of an anti-IL-23p19 antibody for the manufacture of medicament for use in reducing bowel urgency in a patient having or suspected of having ulcerative colitis, wherein the treatment comprises obtaining a first sample from the patient; analyzing the first sample to detect at least one biomarker selected from C-reactive protein (CRP) and fecal calprotectin (fCLP); administering the anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

In an embodiment, the sample is whole blood, plasma, serum, tissue biopsy, fecal samples, and combinations thereof.

In another embodiment, the sample is a combination of a serum sample and a fecal sample, wherein CRP is detected in the serum sample and fCLP is detected in the fecal sample.

In another embodiment, CRP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, or combinations thereof, and fCLP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunonephelometry assay, immunoprecipitation, immunohistochemistry, or combinations thereof.

In another embodiment, CRP is detected in a serum sample by a CRP HS immunonephelometry assay and fCLP is detected in a fecal samples by an ELISA.

In another embodiment, the first sample is taken before or simultaneous with administration of the anti-IL-23p19 antibody, and wherein the second sample is taken at least two weeks, at least four weeks, at least eight weeks, at least twelve weeks, at least sixteen weeks, at least twenty weeks, at least twenty-four weeks, at least twenty-eight weeks, at least thirty weeks, at least thirty-two weeks, at least thirty-six weeks, at least forty weeks, at least forty-four weeks, at least forty-eight weeks, or at least fifty-two weeks, after the first administration of the anti-IL-23p19 antibody.

In another embodiment, the anti-IL-23p19 antibody is mirikizumab, guselkumab, risankizumab, tildrakizumab or brazikumab.

In a preferred embodiment, the anti-IL-23p19 antibody is mirikizumab.

In another embodiment, administering mirikizumab to the patient comprises:
a) administering at least one induction dose of mirikizumab to the patient, wherein the induction dose comprises 50 mg to 600 mg of mirikizumab; and
b) administering at least one maintenance dose(s) of mirikizumab to the patient after the last induction dose is administered, wherein the maintenance dose comprises 150 to 400 mg of mirikizumab.

In another embodiment, the induction doses of mirikizumab are administered about every 4 weeks for about 12 weeks.

In another embodiment, the induction dose(s) of mirikizumab is administered intravenously.

In another embodiment, the at least one maintenance dose is administered subcutaneously.

In another embodiment, wherein the method/treatment comprises:
a) administering three induction doses of mirikizumab to the patient by intravenous infusion at 4-week intervals, wherein each induction dose comprises 300 mg of mirikizumab; and
b) administering maintenance doses of mirikizumab to the patient by subcutaneous injection at 4 week or 12 week intervals, wherein the first maintenance dose is administered 2-8 weeks after the last induction dose is administered and wherein each maintenance dose comprises 200 mg of mirikizumab.

In another embodiment, the first maintenance dose is administered 4-6 weeks after the last induction dose is administered.

In another embodiment, subsequent maintenance dose(s) of mirikizumab are administered at 4-week intervals after administration of the first maintenance dose.

In another embodiment, subsequent maintenance dose(s) of mirikizumab are administered at 12-week intervals after administration of the first maintenance dose.

In an alternative preferred embodiment, the anti-IL-23p19 antibody is guselkumab.

In another embodiment, the method comprises:
a) administering three induction doses of guselkumab to the patient by intravenous infusion at 4-week intervals, wherein each induction dose comprises 100-500 mg of guselkumab; and
b) administering maintenance doses of guselkumab to the patient by subcutaneous injection at 2-week, 4-week, 6-week, 8-week or 12-week intervals, wherein the first maintenance dose is administered 2-8 weeks after the last induction dose is administered.

In another embodiment, each induction dose comprises 200 mg of guselkumab.

In another embodiment, each induction dose comprises 400 mg of guselkumab.

In another embodiment, the anti-IL-23p19 antibody is risankizumab.

In another embodiment, the anti-IL-23p19 antibody is tildrakizumab.

In another embodiment, the anti-IL-23p19 antibody is brazikumab.

The methods can further include analyzing at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

Symptoms of ulcerative colitis include at least one of abdominal pain/discomfort, blood in stool, pus in stool, fever, weight loss, rectal bleeding, frequent diarrhea, recurrent diarrhea, fatigue, reduced appetite, and tenesmus (urgency).

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of determining whether bowel urgency in a patient having or suspected of having ulcerative colitis is reduced in response to anti-IL-23p19 antibody treatment. The method includes analyzing a sample obtained from a patient before the patient receives anti-IL-23p19 antibody treatment for at least one biomarker including C-reactive protein (CRP) and fecal calprotectin (fCLP); analyzing a sample obtained from a patient after the patient receives the anti-IL-23p19 antibody treatment for at least one biomarker including C-reactive protein (CRP) and fecal calprotectin (fCLP); and determining that bowel urgency is reduced in the patient having or suspected of having ulcerative colitis in response to the anti-IL-23p19 antibody treatment if a change in expression level in the at least one biomarker after the patient receives the anti-IL-23p19 antibody treatment is detected.

In another embodiment, the sample is whole blood, plasma, serum, tissue biopsy, fecal samples, and combinations thereof.

In another embodiment, CRP is detected in the serum sample and fCLP is detected in the fecal sample.

In another embodiment, CRP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, or combinations thereof, and fCLP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunonephelometry assay, immunoprecipitation, immunohistochemistry, or combinations thereof.

In another embodiment, CRP is detected in a serum sample by a CRP HS immunonephelometry assay and fCLP is detected in a fecal samples by an ELISA.

In another embodiment, the first sample is taken before or simultaneous with administration of the anti-IL-23p19 antibody, and wherein the second sample is taken at least two weeks, at least four weeks, at least eight weeks, at least twelve weeks, at least sixteen weeks, at least twenty weeks, at least twenty-four weeks, at least twenty-eight weeks, at least thirty weeks, at least thirty-two weeks, at least thirty-six weeks, at least forty weeks, at least forty-four weeks, at least forty-eight weeks, or at least fifty-two weeks, after the first administration of the anti-IL-23p19 antibody.

In another embodiment, the anti-IL-23p19 antibody is mirikizumab, guselkumab, risankizumab, tildrakizumab or brazikumab.

In a preferred embodiment, the anti-IL-23p19 antibody is mirikizumab.

The method can further include analyzing a sample obtained from a patient having or suspected of having ulcerative colitis who did not receive anti-IL-23p19 antibody treatment for at least one biomarker including C-reactive protein and fecal calprotectin.

The method can further include analyzing at least one biomarker including C-reactive protein and fecal calprotectin, wherein an expression level of at least one of C-reactive protein and fecal calprotectin after anti-IL-23p19 antibody treatment is decreased as compared to an expression level of at least one of C-reactive protein and fecal calprotectin before anti-IL-23p19 antibody treatment in the patient who is administered anti-IL-23p19 antibody.

The method can further include analyzing an expression level of at least one biomarker including C-reactive protein and fecal calprotectin in a patient who is not administered an anti-IL-23p19 antibody; comparing the expression level of the at least one biomarker in the patient who is not administered anti-IL-23p19 antibody to an expression level of at least one of C-reactive protein and fecal calprotectin in a patient administered anti-IL-23p19 antibody treatment; and determining that the patient administered anti-IL-23p19 antibody treatment reduces bowel urgency if the biomarker expression level in the patient administered anti-IL-23p19 antibody treatment is reduced as compared to the expression level of the biomarker in the patient who did not receive anti-IL-23p19 antibody treatment.

The methods can further include analyzing at least one biomarker selected from CXCL8, AQP9, ILIB, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

In another aspect, the present disclosure is directed to a biomarker panel. The biomarker panel includes at least one biomarker including C-reactive protein and fecal calprotectin.

### Examples

### Materials and Methods

### Study Design and Participants

Urgency was assessed as part of a multicenter, randomized, double-blind, parallel-arm, placebo-controlled trial. The trial was conducted at 75 sites in 14 countries. Patients were enrolled from January 2016 to September 2017.

The study was compliant with the International Conference on Harmonisation (ICH) guideline on good clinical practice. All informed consent forms and protocols were approved by appropriate ethical review boards prior to initiation of the study. All patients gave written informed consent prior to receiving the study drug.

### Procedures and Outcomes

Patients reported daily symptoms in an electronic diary, including patient-assessed bowel urgency status (absence or presence of bowel urgency).

Induction treatment consisted of 50, 200 or 600 mg of mirikizumab or PBO administered IV at Weeks 0, 4, and 8. At Week 12, patients who achieved clinical response (a decrease in 9-point modified Mayo score [rectal bleeding, stool frequency, and endoscopy] inclusive of ≥2 points and >35% from baseline with either a decrease of rectal bleeding subscore of ≥1 or a rectal bleeding subscore of 0 or 1) were eligible to enter the maintenance period, where they were re-randomized to receive mirikizumab 200 mg subcutaneously (SC) every 4 weeks (Q4W) or every 12 weeks (Q12W). To maintain the blind of the study, patients who received PBO in the induction period and achieved clinical response at Week 12 continued to receive PBO SC Q4W in a treat-through study design. There was no randomized withdrawal to PBO in maintenance.

Fecal calprotectin was measured in patient-collected fecal samples by using an enzyme immunoassay by Bühlmann Laboratories (Schönenbuch, Switzerland) and tested by Covance Central Labs (Princeton, NJ). CRP was measured in collected serum samples using a CRP HS immunonephelometry assay (Siemens BN II; Siemens Healthineers, Munich, Germany) performed at Covance Central.

### Outcomes

The first outcome measure for this study was patient-reported bowel urgency status. Absence of urgency at Weeks 12 and 52 was defined as reporting no urgency for the three consecutive days as recorded in a daily diary prior to each scheduled visit, regardless of bowel urgency status at baseline. Bowel urgency outcomes were defined post-hoc.

Other pre-specified outcome measures included clinical remission (Mayo subscores of 0 for rectal bleeding (RB), 0 or 1 with 1 point decrease from baseline for stool frequency, and 0 or 1 for centrally read endoscopy); clinical response (a decrease in the 9-point modified Mayo subscore [rectal bleeding, stool frequency, and endoscopy] inclusive of ≥2 points and ≥35% from baseline with either a decrease of rectal bleeding subscore of ≥1 or a rectal bleeding subscore of 0 or 1); endoscopic remission (Mayo endoscopic subscore of 0); endoscopic healing (endoscopic subscore of 0 or 1); change from baseline in the Inflammatory Bowel Disease Questionnaire (IBDQ); change from baseline in fecal calprotectin (fCLP) and C reactive protein (CRP). Stool frequency remission and rectal bleeding remission outcomes were derived from their respective component of the clinical remission definition. Symptomatic response and remission are comprised of the RB and SF components of clinical response and remission, respectively. Analyses were performed in all patients regardless of bowel urgency status at baseline.

The IBDQ is a 32-item subject-completed questionnaire that measures 4 aspects of subjects' lives: symptoms directly related to the primary bowel disturbance, systemic symptoms, emotional function, and social function. A total IBDQ score of ≥170 points was considered the threshold for IBDQ remission in the trial, with the Minimal Clinically Important Difference (MCID) defined as an improvement of ≥16 points in the total IBDQ score.

The SF-36 (36-Item Short Form Health Survey version 2) is a 36-item subject-completed measure designed to be a short, multipurpose assessment of health in the areas of physical functioning, role-physical, role-emotional, bodily pain, vitality, social functioning, mental health, and general health. The 2 overarching domains of mental well-being and physical well-being are captured by the mental and physical component summary scores (MCS and PCS, respectively). A ≥5-point improvement (decrease) was defined as the MCID for SF-36 PCS and MCS scores in this trial. The SF-36 has demonstrated validity and reliability when used in UC patients.

### Statistical Analysis

The intention-to-treat (ITT) population, which includes all randomized patients, was used to assess outcomes through Week 12. For the maintenance period, the analysis population consisted of a subset of the ITT population and included those patients who were re-randomized to one of the two maintenance mirikizumab arms or continued on to subcutaneous placebo after demonstrating clinical response at Week 12. All analyses were conducted by pooling together patients in the ITT population across treatment groups for Week 12 analyses, and patients in the ITT population that experienced clinical response at Week 12 for the Week 52 analyses.

Categorical outcome measures were analyzed using a logistic regression analysis with treatment group, geographic region, prior biologic experience (prior biologic experience vs prior biologic naïve), and visit (when appropriate) in the model. Continuous endpoints were analyzed using a Mixed effect Model Repeat Measurement (MMRM) technique with treatment, visit, geographic region, prior biologic experience, treatment-by-visit interaction, and the continuous, fixed covariates of baseline value and baseline value-by-visit interaction terms included in the model. Non-responder imputation (NRI) was utilized for patients who discontinued the study before receiving a Week 12 (induction) or Week 52 (maintenance) endoscopic assessment.

Analysis of covariance (ANCOVA) models were used to compare the change from baseline to Week 12 for each QOL measure (IBDQ and SF-36) and change from baseline in biomarker values against bowel urgency status, with geographic region, prior biologic experience, age, gender, and baseline value of the QoL measure included in the model; the analyses were then repeated for IBDQ and SF-36 with the additional inclusion of rectal bleeding and stool frequency in the model, and Type III squared partial correlation coefficients were calculated from this model to compare the proportion of variation in QoL gained from adding each symptom to the model; this was invariant for both categorical and continuous variables.

Similar models were used to examine the change from baseline to Week 52 among the ITT population that showed clinical response and are continued to the maintenance period. Type III tests for the Least square means were used for the statistical comparisons between treatment groups. Patients with no urgency data (Week 12 n=9, Week 52 n=6) were imputed as having experienced urgency. Modified baseline observation carried forward (mBOCF) was used to impute missing QOL values: for patients discontinuing due to an AE, the baseline observation was carried forward to the corresponding endpoint for evaluation. For patients discontinuing for any other reason, the last non-missing postbaseline observation before discontinuation was carried forward to the corresponding endpoint for evaluation.

Logistic regression models were used to assess the association between achieving clinical remission, response, or individual components of Mayo remission and absence or presence of urgency at Weeks 12 and 52, with geographic region, prior biologic experience, age, and gender included in the model.

To further assess the association between achieving absence of urgency and improvement in other symptoms (SF and RB), we compared the time to symptomatic remission and response between patients with and without absence of urgency at baseline and Weeks 4, 8, and 12 using Kaplan-Meier curves and log-rank tests.

This study was registered with ClinicalTrials.gov, number NCT02589665.

### Results

### Baseline Demographics and Disease Characteristics

Baseline demographics and disease characteristics were grouped by bowel urgency status at Week 12 and Week 52 and included all patients regardless of urgency status at baseline. Bowel urgency was absent in 27/249 (10.8%) of patients at baseline. In general, baseline characteristics, including Mayo score components of stool frequency, rectal bleeding, and endoscopy, were similar in patients with both absence and presence of urgency at Week 12 and Week 52. Patients who had absence of urgency at Week 12 had lower mean disease duration (absence of urgency 6.9±5.7 years, presence of urgency 9.0±9.1 years, p<0.05), higher mean baseline SF-36 PCS and total IBDQ scores (SF-36 PSC: absence of urgency 43.9±6.8, presence of urgency 40.8±7.3; IBDQ: absence of urgency 133.6±33.7 presence of urgency 121.5±30.2; p<0.05 for both), and were less likely to have urgency at baseline (absence of urgency: 79% urgency at BL, presence of urgency: 95% urgency at BL, p<0.05) compared to those with urgency at Week 12. Those who achieved absence of urgency at Week 52 had lower mean weight (absence of urgency 71.3±16.6 kg, presence of urgency 78.7±18.5 kg, p<0.05) and higher mean total IBDQ scores (absence of urgency 128.9±30.3, presence of urgency 115.7±28.0, p<0.05) at baseline compared to those with urgency at Week 52 (Table 1).

TABLE 1
Baseline demographics and disease characteristics grouped by bowel urgency status at Week 12 or Week 52.

| | Week 12 Bowel Urgency Status | | | | Week 52 Bowel Urgency Status | | | |
|---|---|---|---|---|---|---|---|---|
| | Bowel Urgency | | Bowel Urgency | | Bowel Urgency | | Bowel Urgency | |
| Mean (SD) unless | absent | | present | | absent | | present | |
| otherwise noted | N = 91 | | N = 158 | | N = 66 | | N = 40 | |
| Age, years | 41.1 | (13.3) | 43.4 | (14.2) | 39.6 | (12.8) | 41.6 | (15.0) |
| Weight, kg | 73.7 | (16.7) | 75.7 | (16.7) | 71.3 | (16.6)* | 78.7 | (18.5) |
| Disease | 6.9 | (5.7)* | 9.0 | (9.1) | 8.3 | (6.8) | 6.9 | (6.6) |
| duration, years | | | | | | | | |
| Prior biologic | 46 | (51) | 107 | (68) | 32 | (48) | 22 | (55) |
| use, n (%) | | | | | | | | |
| Concomitant | | | | | | | | |
| therapies - n (%) | | | | | | | | |
| 5-ASA | 66 | (73) | 118 | (75) | 54 | (82) | 31 | (78) |
| Corticosteroids | 44 | (48) | 78 | (49) | 28 | (42) | 20 | (50) |
| Thiopurines | 23 | (25) | 46 | (29) | 20 | (30) | 9 | (23) |
| Bowel Urgency | 72 | (79)* | 150 | (95) | 59 | (89) | 36 | (90) |
| at BL, n (%) | | | | | | | | |
| Mayo score, | | | | | | | | |
| n (%) | | | | | | | | |
| 6-8 | 45 | (50) | 59 | (38) | 25 | (38) | 20 | (50) |
| 9-12 | 45 | (50) | 98 | (62) | 41 | (62) | 20 | (50) |
| Mayo SF | 2.2 | (0.8) | 2.5 | (0.7) | 2.4 | (0.8) | 2.3 | (0.8) |
| subscore | | | | | | | | |
| Mayo RB | 1.4 | (0.9) | 1.4 | (0.8) | 1.6 | (0.8) | 1.4 | (0.8) |
| subscore | | | | | | | | |
| Mayo | 2.7 | (0.5) | 2.8 | (0.4) | 2.7 | (0.5) | 2.7 | (0.5) |
| Endoscopic | | | | | | | | |
| subscore | | | | | | | | |
| SF-36 | | | | | | | | |
| Physical | 43.9 | (6.8)* | 40.8 | (7.3) | 42.5 | (8.3) | 40.7 | (6.9) |
| Component | | | | | | | | |
| Score | | | | | | | | |
| Mental | 40.4 | (10.1) | 38.7 | (10.7) | 39.7 | (9.6) | 36.8 | (10.1) |
| Component | | | | | | | | |
| Score | | | | | | | | |
| IBDQ total | 133.6 | (33.7)* | 121.5 | (30.2) | 128.9 | (30.3)* | 115.7 | (28.0) |
| score | | | | | | | | |
| CRP, mg/L, | 3.3 | (0.1-164.0) | 5.1 | (0.1-77.1) | 3.3 | (0.1-84.9) | 6.7 | (0.1-164.0) |
| median (range) | | | | | | | | |
| fCLP, mg/kg, | 1700.5 | (15.0-31680) | 1442.0 | (15.0-22742) | 1576.0 | (15.0-31680) | 1773.0 | (99.0-10241) |
| median (range) | | | | | | | | |

Baseline demographics and disease characteristics at Week 0, grouped by urgency status at Week 12 or Week 52;
*p < 0.05 compared to those with presence of urgency at Week 12 or 52;
ASA = aminosalicylic acid;
IBDQ = Inflammatory Bowel Disease Questionnaire;
SF-36 = Medical Outcomes Study 36-Item Short Form Health Survey Version 2 Standard;
CRP: C reactive protein;
fCLP: fecal calprotectin

### IBDQ

Total IBDQ score and the percent change from baseline (% ABL) in total IBDQ score was significantly greater in patients who achieved absence of urgency at Week 12 compared to those who had urgency. A similar pattern was observed for the 4 IBDQ subscores, which corresponded to percent changes from BL (% ABL) that were more than two times greater in patients with absence of urgency compared to patients with presence of urgency. These differences in IBDQ scores remained highly significant when adjusted for RB and SF, indicating that bowel urgency is significantly associated with IBD-related quality of life independent of disease activity (Table 2).

TABLE 2
Change from baseline in quality of life measures in patients with absence or
presence of bowel urgency, adjusted for rectal bleeding and stool frequency.

| | Week 12 | | | |
|---|---|---|---|---|
| | Bowel Urgency | Bowel Urgency | Difference in | |
| | Absent | Present | LSM ΔBL | |
| LSM ΔBL (SE) | N = 91 | N = 156 | (95% CI) | P-value |
| IBDQ Total | 25.3 (4.6) | 11.3 (3.9) | 14.0 (5.9, 22.1) | <0.001 |
| score | | | | |
| IBDQ Bowel | 10.0 (1.4) | 5.0 (1.2) | 4.9 (2.6, 7.3) | <0.001 |
| Symptoms | | | | |
| IBDQ Emotional | 8.3 (2.1) | 3.7 (1.7) | 4.6 (1.1, 8.2) | 0.011 |
| Function | | | | |
| IBDQ Social | 3.2 (1.0) | 0.8 (0.8) | 2.4 (0.6, 4.2) | 0.008 |
| Function | | | | |
| IBDQ Systemic | 3.8 (0.8) | 2.1 (0.7) | 1.7 (0.3, 3.2) | 0.019 |
| Symptoms | | | | |
| SF-36 MCS | 3.2 (1.6) | 1.1 (1.3) | 2.2 (−0.6, 4.9) | 0.118 |
| SF-36 PCS | 4.6 (1.0) | 3.2 (0.9) | 1.3 (−0.4, 3.1) | 0.142 |
| | Week 52 | | | |
| | Bowel Urgency | Bowel Urgency | Difference in | |
| | Absent | Present | LSM ΔBL | |
| LSM ΔBL (SE) | N = 66 | N = 40 | (95% CI) | P-value |
| IBDQ Total | 43.5 (8.3) | 28.0 (7.7) | 15.5 (5.0, 25.9) | 0.004 |
| score | | | | |
| IBDQ Bowel | 16.0 (2.4) | 10.9 (2.2) | 5.2 (2.2, 8.1) | <0.001 |
| Symptoms | | | | |
| IBDQ Emotional | 12.5 (3.7) | 8.6 (3.4) | 3.9 (−0.7, 8.5) | 0.096 |
| Function | | | | |
| IBDQ Social | 7.5 (1.8) | 4.8 (1.7) | 2.7 (0.3, 5.0) | 0.025 |
| Function | | | | |
| IBDQ Systemic | 6.7 (1.6) | 3.3 (1.5) | 3.4 (1.4, 5.4) | 0.001 |
| Symptoms | | | | |
| SF-36 MCS | 3.7 (3.4) | 2.8 (3.2) | 0.9 (−3.3, 5.2) | 0.668 |
| SF-36 PCS | 8.3 (2.0) | 5.4 (1.9) | 2.9 (0.3, 5.5) | 0.026 |

LSM = least squares mean;
SE = standard error;
ΔBL = change from BL (Week 0);
IBDQ = Inflammatory Bowel Disease Questionnaire;
MCS = mental component score;
PCS = physical component score;
CI = confidence interval

### SF-36

As with IBDQ scores, the SF-36 mental component score (MCS) and physical component scores (PCS) as well as the % ABL in both MCS and PCS were significantly higher in patients with absence of urgency at Week 12. However, once these scores were adjusted for RB and SF, only the Week 52 PCS was significantly higher in patients with absence of urgency (Table 2).

### Correlation Between Urgency, RB, and SF and IBDQ at Week 12

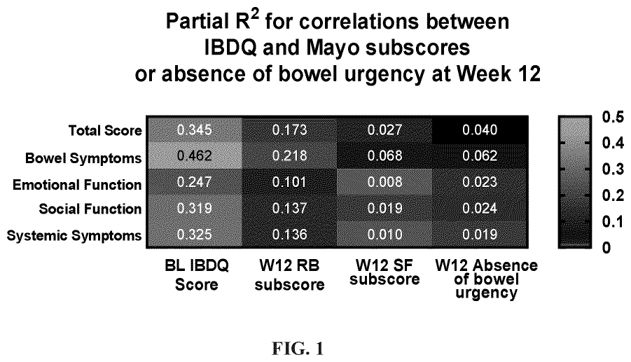

Type III squared partial correlation coefficients were calculated for IBDQ and symptom components (RB, SF, absence of urgency) at Week 12 using an ANCOVA model that included baseline IBDQ score, and Week 12 urgency status, RB subscore, and SF subscore (FIG. 1) in order to compare the contribution of each symptom towards variance in QoL. As expected, BL IBDQ score was most closely correlated with total IBDQ score at Week 12. Of the three symptoms, RB was the most closely correlated with IBDQ, with partial R2 values ranging from 0.101 for emotional function to 0.218 for bowel symptoms. While lower than those of RB, the partial R2 values for absence of bowel urgency were higher than those of SF in every category except bowel symptoms, where the magnitudes of the partial R2 were similar; this suggests that bowel urgency was as important of a contributor, if not more so, to improvements in IBDQ as SF.

### Inflammatory Biomarkers

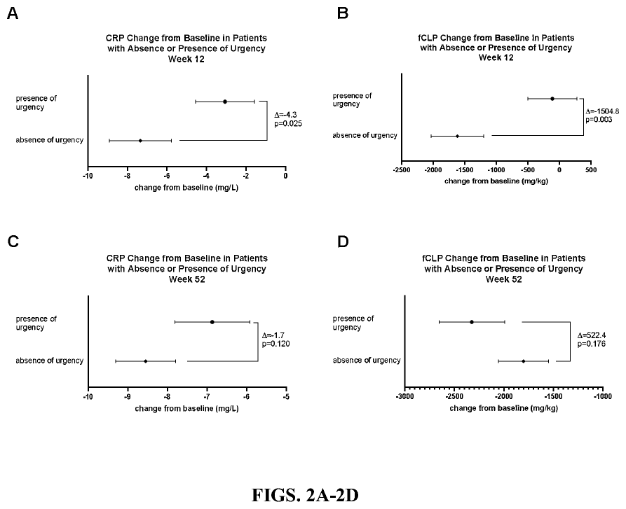

Δt Week 12, patients with absence of urgency had significantly greater ABL in both CRP and fCLP compared to patients with presence of urgency (CRP: absence of urgency-7.34±1.57; presence of urgency −3.06±1.49, p=0.025; fCLP: absence of urgency −1614.07±415.54; presence of urgency −109.24±388.35, p=0.003). At Week 52, although there was no statistically significant difference in ABL in CRP or fCLP between patients with absence or presence of urgency, levels of CRP and fCLP continued to decrease from Week 12 to Week 52 (Table 3, FIG. 2).

TABLE 3
CRP or fCLP level by urgency status

| | Week 0 | | | |
|---|---|---|---|---|
| | Bowel Urgency absent | | Bowel Urgency present | |
| | N = 27 (CRP), 27 (fCLP) | | N = 220 (CRP), 203 (fCLP) | |
| CRP, mg/L, median (range) | 1.9 | (0.1-30.2) | 4.9 | (0.1-164.0) |
| fCLP, mg/kg, median | 1293.0 | (99.0-31680.0) | 1584.0 | (15.0-31680.0) |
| (range) | | | | |
| | Week 12 | | | |
| | Bowel Urgency absent | | Bowel Urgency present | |
| | N = 90 (CRP), 85 (fCLP) | | N = 144 (CRP), 130 (fCLP) | |
| CRP, mg/L, median (range) | 1.8 | (0.1-43.1) | 4.1 | (0.1-138.0) |
| fCLP, mg/kg, median | 252.0 | (15.0-15671.0) | 1019.0 | (15.0-19286.0) |
| (range) | | | | |

| | Week 52 | | | |
|---|---|---|---|---|
| | Bowel Urgency absent | | Bowel Urgency present | |
| | N = 64 (CRP), 59 (fCLP) | | N = 30 (CRP), 27 (fCLP) | |
| CRP, mg/L, median (range) | 1.5 | (0.1-21.3) | 3.0 | (0.1-22.7) |
| fCLP, mg/kg, median | 173.0 | (15.0-12984.0) | 239.0 | (15.0-2100.0) |
| (range) | | | | |

CRP: C reactive protein;
fCLP: fecal calprotectin

### Clinical Outcomes Week 12

Δt Week 12, patients with absence of urgency had significantly higher rates of improvement in all clinical outcomes examined compared to those with presence of urgency (Table 4) (Clinical remission: 22/91 [24.2%] of patients with absence of urgency vs 12/158 [7.6%] in those with presence of urgency [p=0.0019]; clinical response: 64/91 [70.3%] of patients with absence of urgency vs 42/158 [26.6%] in those with presence of urgency [p<0.0001]; stool frequency remission: 62/91 [68.1%] of patients with absence of urgency vs 48/158 [30.4%] in those with presence of urgency [p<0.0001]; rectal bleeding remission: 74/91 [81.3%] of patients with absence of urgency vs 57/158 [36.1%] in those with presence of urgency [p<0.0001]; endoscopic healing: 29/91 [31.9%] of patients with absence of urgency vs 17/158 [10.8%] in those with presence of urgency [p=0.0004]). Of the patients who achieved clinical remission at Week 12, 35% were still experiencing urgency, while 37% of the patients who achieved endoscopic healing were still experiencing urgency.

TABLE 4
Clinical outcome measures in patients with absence or presence of bowel urgency.

| | Week 12 | | | |
|---|---|---|---|---|
| | Bowel Urgency | Bowel Urgency | | Adjusted odds |
| | Absent | Present | P- | ratio |
| All values n (%) | (N = 91) | (N = 158) | value^{a} | (95% CI) |

| | | | | | |
|---|---|---|---|---|---|
| Clinical remission^{b} | 22 (24.2) | 12 (7.6) | 0.0019 | 3.6 | (1.6, 8.0) |
| Clinical response^{c} | 64 (70.3) | 42 (26.6) | <0.0001 | 6.3 | (3.4, 11.6) |
| Mayo stool frequency | 62 (68.1) | 48 (30.4) | <0.0001 | 4.6 | (2.6, 8.2) |
| remission^{d} | | | | | |
| Mayo rectal bleeding | 74 (81.3) | 57 (36.1) | <0.0001 | 7.2 | (3.8, 13.7) |
| remission^{e} | | | | | |
| Mayo endoscopic healing^{f} | 29 (31.9) | 17 (10.8) | 0.0004 | 3.6 | (1.8, 7.2) |

| | Week 52 | | | | |
|---|---|---|---|---|---|
| | Bowel Urgency | Bowel Urgency | | | |
| | Absent | Present | | Adjusted odds | |
| | N = 66 | N = 40 | P value | ratio | |
| Clinical remission | 35 (53.0) | 5 (12.5) | <.0001 | 10.7 | (3.3, 35.0) |
| Clinical response | 62 (93.9) | 19 (47.5) | <.0001 | 24.0 | (6.2, 92.5) |
| Mayo stool frequency | 60 (90.9) | 16 (40.0) | <.0001 | 19.8 | (5.7, 68.1) |
| remission | | | | | |
| Mayo rectal bleeding | 59 (89.4) | 22 (55.0) | 0.0004 | 6.6 | (2.3, 18.6) |
| remission | | | | | |
| Mayo endoscopic remission | 40 (60.6) | 11 (27.5) | 0.0005 | 5.6 | (2.1, 14.7) |

^{a}From logistic regression model for remissions or responses, adjusted by urgency status, age, sex, geographic region and prior biologic experience. Missing values for the clinical outcome and absence of urgency were imputed using non-responder imputation;
^{b}Clinical remission: RB Mayo subscore of 0, SF Mayo subscore of 0 or 1 with a >=1 point decrease from baseline, and Mayo endoscopic subscore of 0 or 1;
^{c}Clinical response: a decrease in the 9-point Mayo subscores (comprising the subscores of rectal bleeding, stool frequency and the endoscopic findings) inclusive of >=2 points and >=35% from baseline with either a decrease of rectal bleeding subscore of >=1 or rectal bleeding subscore of 0 or 1;
^{d}Stool frequency remission: Mayo SF = 0, or 1 with a >=1 point decrease from baseline;
^{e}Rectal bleeding remission: Mayo RB = 0;
^{f}Endoscopic healing: Mayo endoscopy = 0 or 1

### Clinical Outcomes Week 52

Similarly, absence of urgency at Week 52 was associated with significantly higher rates of improvement in all clinical outcomes examined at Week 52 (Table 4). (Clinical remission: 35/66 [53.0%] of patients with absence of urgency, 5/40 [12.5%] in those with presence of urgency [p<0.0001]; clinical response: 62/66 [93.9%] of patients with absence of urgency, 19/40 [47.5%] in those with presence of urgency [p<0.0001]; stool frequency remission: 60/66 [90.9%] of patients with absence of urgency, 16/40 [40.0%] in those with presence of urgency [p<0.0001]; rectal bleeding remission: 59/66 [89.4%] of patients with absence of urgency, 22/40 [55.0%] in those with presence of urgency [p=0.0004]; endoscopic healing: 40/66 [60.6%] of patients with absence of urgency, 11/40 [27.5%] in those with presence of urgency [p=0.0005]). At Week 52, the proportion of patients in clinical remission who were still experiencing urgency decreased to 5%, while those with endoscopic healing who were still experiencing urgency decreased to 22%.

### Association Between Bowel Urgency and Time to Symptomatic Response or Remission

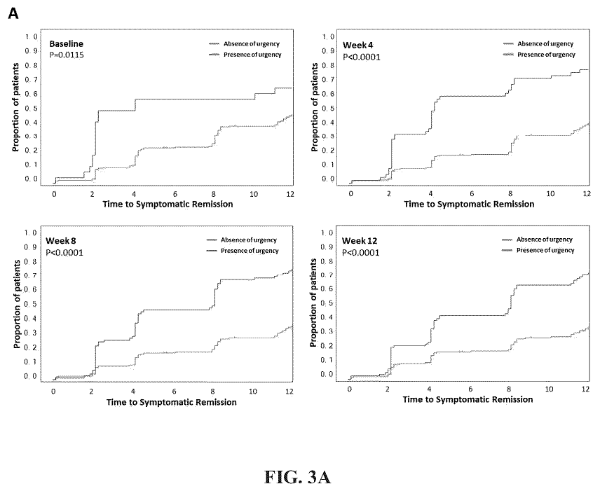

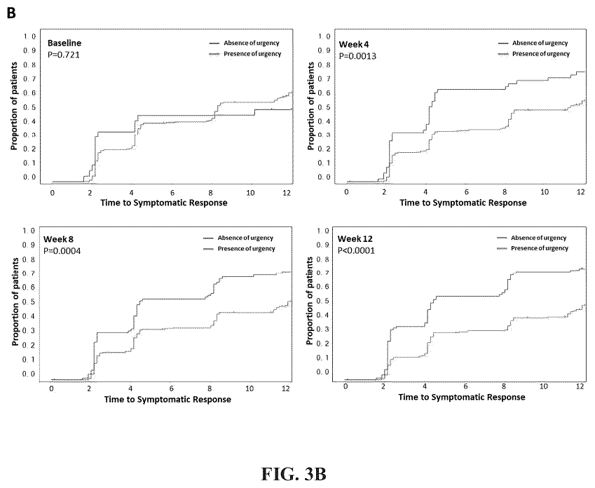

A Kaplan-Meier analysis showed that patients who report absence of urgency at baseline, or achieve absence of urgency at Weeks 4, 8, or 12 had a faster time to symptomatic remission than patients still experiencing bowel urgency at those time points (FIG. 3a). Likewise, patients who achieve absence of urgency at Weeks 4, 8, or 12 had a faster time to symptomatic response (FIG. 3b) than those with presence of urgency.

### Discussion

In this analysis of the results of a Phase 2 study of mirikizumab in patients with UC, we show that the absence of bowel urgency is associated with improved QOL measures, decreased levels of inflammatory biomarkers, and improved clinical outcomes. When adjusted for disease activity (e.g. stool frequency and rectal bleeding), this association remained strong for all IBDQ subcategories, indicating that this is an independent effect of bowel urgency. This was not the case for SF-36, however, most likely because SF-36 is a generic health quality of life survey and thus may not directly address bowel urgency, while IBDQ is specific for IBD. Among the IBDQ subcategories, absence of urgency correlated most strongly with improved bowel symptoms at Week 12, followed by improved social function, while at Week 52 absence of urgency correlated most strongly with improved bowel symptoms followed by improved systemic symptoms. Absence of urgency was strongly associated with all clinical outcomes that were measured. Notably and impressively, at Week 52, of the patients in clinical remission, only 5% reported urgency.

Absence of urgency was strongly associated with reduced levels of both CRP and fCLP after 12 weeks of induction treatment with mirikizumab, suggesting a reduction in colonic inflammation associated with absence of urgency. Among the induction responder subgroup of patients, who all received mirikizumab through Week 52, levels of biomarkers continued to decrease in patients with both presence and absence of urgency through Week 52, demonstrating that in the Week 12 responder population inflammation lowers over time regardless of the presence of urgency, but a greater initial drop is associated with absence of urgency. The unpredictable and uncontrollable nature of bowel urgency can result in a great deal of anxiety, panic, and stress for patients with UC, and there may be a connection between anxiety and bowel urgency (e.g., urgency related “PTSD”) independent of inflammation.

Patients who achieved absence of urgency at Week 12 had significantly shorter disease duration and higher baseline total IBDQ scores compared to those with presence of urgency at Week 12, as well as a greater proportion of patients with absence of urgency at baseline. Neither disease duration nor presence of bowel urgency at baseline was significantly different between patients who achieved absence of urgency at Week 52 and those who did not. Thus, disease duration higher QOL as measured by the IBDQ may be an early indicator of rapid treatment-induced resolution of bowel urgency, but not necessarily of long-term response. Interestingly, disease activity, as measured by Mayo score components, was not significantly different at baseline in patients with or without urgency.

The presence of bowel urgency has previously been reported to have significant impact on patient quality of life, limiting participation in physical and social activities, impinging on the ability to travel to and from work, and causing significant stress, anxiety, feelings of social isolation, and stigma. The findings that all IBDQ domain scores were significantly improved in patients with absence of urgency compared to those with presence of urgency, even after adjusting for disease activity, are in agreement with and further support these prior studies, demonstrating the strong impact that bowel urgency has on quality of life in patients with UC and suggesting that lack of bowel urgency may be used as a rough surrogate for QoL measures.

The percentage of patients achieving a given clinical outcome was strongly associated with absence of urgency in patients for all clinical outcomes that were assessed. A number of patients with clinical remission or endoscopic healing (and thus no rectal bleeding and normal stool frequency) still experienced bowel urgency however, indicating that this association was independent of standard methods to assess disease activity. Moreover, our analyses show that earlier resolution of bowel urgency is associated with shorter time to symptomatic response and remission (FIG. 3). Given the association of urgency improvement with rapid treatment response as well as with changes in inflammatory markers, bowel urgency could potentially be used by clinicians along with rectal bleeding and stool frequency as surrogate markers to guide communication to patients about expected rapidity of treatment response.

While there remains a communication gap between health care providers and patients regarding bowel urgency, this study, has reinforced the importance of bowel urgency from a clinical perspective. Because of the importance of bowel urgency to patients, recommendations have been made to include bowel urgency in the clinical assessment of IBD patients and considered for inclusion in treatment plans. Recent ΔCG Guidelines have stated that initial treatment of UC should focus on restoration of normal stool frequency and control of the primary symptoms of bleeding and urgency. The high correlation we observed between bowel urgency status and both quality of life and clinical outcomes supports the importance of evaluating bowel urgency in a clinical setting, and the inclusion of resolution of bowel urgency as a treatment goal.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

1. A method of treating bowel urgency in a patient having ulcerative colitis, the method comprising: obtaining a first sample from the patient; analyzing the first sample to detect at least one biomarker selected from C-reactive protein (CRP) and fecal calprotectin (fCLP); administering an anti-IL-23p19 antibody to the patient; obtaining a second sample from the patient; and analyzing the second sample to detect at least one biomarker selected from C-reactive protein (CRP) and fecal calprotectin (fCLP), wherein a change in expression level of the at least one biomarker detected in the second sample from the expression level of the at least one biomarker detected in the first sample indicates a response to the anti-IL-23p19 antibody.

2. A method of treating bowel urgency in a patient having ulcerative colitis according to claim 1, wherein the sample is whole blood, plasma, serum, tissue biopsy, fecal samples, and combinations thereof.

3. A method of treating bowel urgency in a patient having ulcerative colitis according to claim 2, wherein the sample is a combination of a serum sample and a fecal sample, wherein CRP is detected in the serum sample and fCLP is detected in the fecal sample.

4. A method of treating bowel urgency in a patient having ulcerative colitis according to claim 1, wherein CRP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, or combinations thereof, and fCLP is detected in the sample by microarray analysis, amplification (polymerase chain reaction), northern blot, southern blot, in situ hybridization, immunoassays including western blot, enzyme-linked immunosorbent assay (ELISA), enzyme-linked fluorescence assay (ELFA), immunonephelometry assay, immunoprecipitation, immunohistochemistry, or combinations thereof.

5. A method of treating bowel urgency in a patient having ulcerative colitis according to claim 1, wherein CRP is detected in a serum sample by a CRP HS immunonephelometry assay and fCLP is detected in a fecal samples by an ELISA.

6. A method of treating bowel urgency in a patient having ulcerative colitis according to claim 1, wherein the first sample is taken before or simultaneous with administration of the anti-IL-23p19 antibody, and wherein the second sample is taken at least two weeks, at least four weeks, at least eight weeks, at least twelve weeks, at least sixteen weeks, at least twenty weeks, at least twenty-four weeks, at least twenty-eight weeks, at least thirty weeks, at least thirty-two weeks, at least thirty-six weeks, at least forty weeks, at least forty-four weeks, at least forty-eight weeks, or at least fifty-two weeks, after the first administration of the anti-IL-23p19 antibody.

7. A method of treating bowel urgency in a patient having ulcerative colitis according to claim 1, wherein the anti-IL-23p19 antibody is mirikizumab, guselkumab, risankizumab, tildrakizumab or brazikumab.

8. A method of treating bowel urgency in a patient having ulcerative colitis according to claim 7, wherein the anti-IL-23p19 antibody is mirikizumab.

9.-12. (canceled)

13. A method of treating bowel urgency in a patient having ulcerative colitis according to claim 8, wherein the method comprises:
a) administering three induction doses of mirikizumab to the patient by intravenous infusion at 4-week intervals, wherein each induction dose comprises 300 mg of mirikizumab; and
b) administering maintenance doses of mirikizumab to the patient by subcutaneous injection at 4 week or 12 week intervals, wherein the first maintenance dose is administered 2-8 weeks after the last induction dose is administered and wherein each maintenance dose comprises 200 mg of mirikizumab.

14. A method of treating bowel urgency in a patient having ulcerative colitis according to claim 13, wherein the first maintenance dose is administered 4-6 weeks after the last induction dose is administered.

15. A method of treating bowel urgency in a patient having ulcerative colitis according to claim 13, wherein subsequent maintenance dose(s) of mirikizumab are administered at 4-week intervals after administration of the first maintenance dose.

16. A method of treating bowel urgency in a patient having ulcerative colitis according to claim 13, wherein subsequent maintenance dose(s) of mirikizumab are administered at 12-week intervals after administration of the first maintenance dose.

17.-23. (canceled)

24. A method of identifying or determining a patient having ulcerative colitis as a candidate patient for receiving an anti-IL-23p19 antibody treatment for bowel urgency, the method comprising: obtaining a sample from the patient; analyzing the sample for at least one biomarker selected from C-reactive protein (CRP) and fecal calprotectin (fCLP); and identifying the patient as a candidate patient for receiving the anti-IL-23p19 antibody treatment for bowel urgency based on the analysis of the at least one biomarker.

25.-30. (canceled)

31. A method for identifying risk for bowel urgency in a patient having ulcerative colitis, wherein the method comprises (a) determining an expression level of at least one biomarker selected from C-reactive protein (CRP) and fecal calprotectin (fCLP) in a sample obtained from the patient; (b) comparing the determined expression level of the at least one biomarker to a reference expression level of at least one biomarker selected from C-reactive protein (CRP) and fecal calprotectin (fCLP); and (c) identifying that the patient is at risk for bowel urgency if the biomarker expression level in the patient is changed as compared to the reference expression level.

32.-112. (canceled)

113. A biomarker panel comprising at least one biomarker selected from C-reactive protein (CRP), fecal calprotectin (fCLP), CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ΔDH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ΔDH1C, PCK1, CDKN2B-AS1, TMEM236, CD177P1, SLC17A4, and ZG16.
